(19) United States
(12) Patent Application Publication
(10) Pub. No.: US 2018/0028125 A1
(43) Pub. Date: Feb. 1, 2018

(54) Health Care System

(71) Applicant: National Kaohsiung University of Applied Sciences, Kaohsiung City (TW)

(72) Inventors: Jian-Chiun Liou, Kaohsiung City (TW); Tien-Tsorng Shih, Kaohsiung City (TW); Cheng-Hong Yang, Kaohsiung City (TW)

(21) Appl. No.: 15/223,004

(22) Filed: Jul. 29, 2016

(51) Int. Cl. A61B 5/00; A61B 5/0488; A61B 5/0408

(52) U.S. Cl. CPC A61B 5/7246; A61B 5/04085; A61B 5/0488; A61B 5/742; A61B 5/0006; A61B 5/0022

(57) ABSTRACT

A health care system including a cloud database, a physiological sensor and a processor is disclosed. The cloud database is configured to store a reference waveform and an abnormal data record. The physiological sensor is configured to detect a detected part of a body and to generate a detected physiological signal. The processor is electrically connected to the cloud database and retrieves the reference waveform from the cloud database. The processor is electrically connected to the physiological sensor to receive the detected physiological signal, converts the detected physiological signal into a physiological waveform, compares the physiological waveform with the reference waveform to determine a frequency error and a peak error therebetween, and generates a driving signal when the frequency error is not smaller than a frequency threshold and the peak error is not smaller than a peak threshold. Advantageously, the efficiency of health management can be improved.

### BACKGROUND OF THE INVENTION

### 1. Field of the Invention

The present disclosure generally relates to a health care system and, more particularly, to a health care system with long-distance data access that determines whether a physiological signal is abnormal through data comparison.

### 2. Description of the Related Art

Due to the growing health consciousness, people are more and more concerned about their health management. In the health self-management, it is very important to keep a track on the daily body condition in order to find out any potential disease at the early stage. This can prevent worsening of the illness condition.

To observe the daily body condition, a health management device is usually used. In general, the health management device includes a display and a physiological sensor. When the user wears the physiological sensor on a certain part of the body, the physiological sensor can detect the physiological signal of the user. The detected physiological signal is transmitted to the display in order to inform the user of his/her physiological condition, including the body temperature, the pulse or the blood pressure.

A conventional health management device is usually not able to determine whether the physiological signal is abnormal or not. Therefore, such a health management device is not able to remind the user of the abnormality of the physiological signal. Although the user can know about his/her body temperature, the pulse or the blood pressure through the health management device, the user is still unable to realize whether his/her body condition is abnormal. Besides, different users have different physiological signals when they are in a healthy condition. Based on this, although the health management device can detect the abnormality of the physiological signal, the health management device is still unable to accurately determine the abnormality of the physiological condition of the user since the health management device has only a single type of reference data for comparison. As a disadvantage, the performance of the conventional health management device is low.

In light of this, it is necessary to provide a health care system that has a high performance.

### SUMMARY OF THE INVENTION

It is therefore the objective of this disclosure to provide a health care system capable of comparing the physiological waveform, which is currently retrieved from a testee, with the reference waveform which was retrieved from the same testee when the testee was in a healthy condition. As such, the health care system can issue a driving signal when there is a large difference between the physiological waveform and the reference waveform.

In an embodiment, a health care system including a cloud database, a physiological sensor and a processor is disclosed. The cloud database is configured to store a reference waveform and an abnormal data record. The physiological sensor is configured to detect a detected part of a body and to generate a detected physiological signal. The processor is electrically connected to the cloud database and retrieves the reference waveform from the cloud database. The processor is electrically connected to the physiological sensor to receive the detected physiological signal, converts the detected physiological signal into a physiological waveform, compares the physiological waveform with the reference waveform to determine a frequency error and a peak error therebetween, generates a driving signal when the frequency error is not smaller than a frequency threshold and the peak error is not smaller than a peak threshold, and transmits the physiological signal to the cloud database as the abnormal data record. As such, the processor can compare the currently-retrieved physiological waveform with the reference waveform previously obtained, and issue the driving signal when there is a large difference between the physiological waveform and the reference waveform. The compared result can be sent to related personnel, increasing the efficiency of health management thereof.

In a form shown, the frequency threshold is substantially 10% of a frequency value of the reference waveform. As such, the processor can issue the driving signal when there is a certain difference between the physiological waveform and the reference waveform. The compared result can be sent to related personnel, increasing the efficiency of health management thereof.

In the form shown, the peak threshold is substantially 10% of a peak value of the reference waveform. As such, the processor can issue the driving signal when there is a certain difference between the physiological waveform and the reference waveform. The compared result can be sent to related personnel, increasing the efficiency of health management thereof.

In the form shown, the physiological sensor is a resistor-type sensor. As such, the physiological sensor can generate the detected physiological signal through the detection of the resistance, attaining an efficient generation of the detected physiological signal.

In the form shown, the resistor-type sensor includes a resistor-type substrate and a metal nanowire array. The resistor-type substrate includes a sensing face. The metal nanowire array is disposed on the sensing face. The metal nanowire array is used to detect a resistance of the detected part of the body, and the detected physiological signal is generated based on the resistance. As such, the physiological sensor can generate the detected physiological signal through the detection of the resistance, attaining an efficient generation of the detected physiological signal.

In the form shown, the physiological sensor is a photoelectric sensor. As such, the physiological sensor can generate the detected physiological signal through the detection of light, attaining an efficient generation of the detected physiological signal.

In the form shown, the photoelectric sensor includes a photoelectric substrate, a light emitting unit and a light detector. The photoelectric substrate includes a surface that is divided into an emission area and a reception area. The light emitting unit is arranged on the emission area and emits at least one light to the detected part of the body. The light detector is arranged on the emission area and is used to receive at least one reflected light reflected from the detected part of the body. The light detector generates the detected physiological signal based on the at least one reflected light. As such, the physiological sensor can generate the detected physiological signal through the detection of light, attaining an efficient generation of the detected physiological signal.

In the form shown, the light emitting unit includes a plurality of light emitting sections capable of emitting a plurality of lights with different wavelengths. The plurality of light emitting sections is arranged on the emission area in intervals along a direction. As such, the light emitting unit can emit different kinds of lights with different wavelengths. When the light emitting unit emits lights towards the detected part of the body, although some part of the lights may not be able to penetrate certain tissue in the body due to its wavelength, another part of the lights is still able to reach the detected part. Thus, the detected physiological signal can be efficiently generated.

In the form shown, the health care system further includes a controller electrically connected to the light emitting unit. The controller is used to control the plurality of light emitting sections to sequentially emit the plurality of lights along the direction. As such, when the plurality of light emitting sections emits different wavelengths of lights to the detected part in sequence, the light detector can sequentially receive the lights reflected from the detected part of the body. Based on this, the light detector can generate the detected physiological signal according to the reflected lights, improving the detection accuracy thereof.

In the form shown, the health care system further includes a controller electrically connected to the light emitting unit. The controller is used to control the plurality of light emitting sections to emit the plurality of lights in a random manner. As such, when the plurality of light emitting sections emits different wavelengths of lights to the detected part in sequence, the light detector can sequentially receive the lights reflected from the detected part of the body. Based on this, the light detector can generate the detected physiological signal according to the reflected lights, improving the detection accuracy thereof.

In the form shown, the quantity of the plurality of light emitting sections is 3, and the plurality of light emitting sections includes a red light emitting section, a green light emitting section and a blue light emitting section. As such, the light emitting unit can emit different kinds of wavelengths of lights. When the light emitting unit emits different wavelengths of lights towards the detected part of the body, although some part of the lights may not be able to penetrate certain tissue in the body due to its wavelength, another part of the lights is still able to reach the detected part. Thus, the detected physiological signal can be efficiently generated.

In the form shown, each of the plurality of light emitting sections includes at least one micro light-emitting diode, and each of the at least one micro light-emitting diode has a size of 20 μm by 20 μm. As such, not only the volume and power consumption of the light emitting unit can be reduced, but also the lights can smoothly reach the detected part due to its finer scale. Thus, the detection accuracy is improved.

In the form shown, the physiological sensor further includes a wireless transmission module, and the processor includes a wireless transceiving module electrically connected to the wireless transmission module. As such, the processor can be electrically connected to the physiological sensor through the wireless transceiving module, improving the convenience in data transmission.

In the form shown, each of the wireless transmission module and the wireless transceiving module is a WIFI structure, a zigbee structure or a Bluetooth structure. As such, the processor can be electrically connected to the physiological sensor through the wireless transceiving module, improving the convenience in data transmission.

In the form shown, the reference waveform and the physiological waveform have a same sensing basis. As such, the processor can compare the currently-retrieved physiological waveform with the reference waveform previously obtained, and can issue the driving signal when there is a large difference between the physiological waveform and the reference waveform. The compared result can be sent to related personnel, increasing the efficiency of health management thereof.

In the form shown, each of the reference waveform and the physiological waveform is an electrocardiogram signal or an electromyogram signal. As such, the processor can compare the currently-retrieved physiological waveform with the reference waveform previously obtained, and can issue the driving signal when there is a large difference between the physiological waveform and the reference waveform. The compared result can be sent to related personnel, increasing the efficiency of health management thereof.

In the form shown, the health care system further includes an electronic device electrically connected to the processor in order to receive the driving signal. The electronic device comprises a warning unit which issues a warning message upon the reception of the driving signal. As such, the warning unit of the electronic device can issue the warning message when there is a large difference between the physiological waveform and the reference waveform. The compared result can be sent to related personnel, increasing the efficiency of health management thereof.

In the form shown, the health care system further includes an electronic device electrically connected to the processor in order to receive the physiological waveform. The electronic device includes a display which is used to display the physiological waveform. As such, the related personnel are able to view the detected result of the physiological sensor via the display, improving the efficiency of health management thereof.

In the form shown, the physiological sensor is a three-lead electrocardiogram signal sensor.

In the form shown, the electronic device is a mobile communication device. As such, the related personnel are able to view the compared result via the electronic device, improving the efficiency of health management thereof.

### BRIEF DESCRIPTION OF THE DRAWINGS

The present disclosure will become more fully understood from the detailed description given hereinafter and the accompanying drawings which are given by way of illustration only, and thus are not limitative of the present disclosure, and wherein:

FIG. 1 shows a block diagram of a health care system according to an embodiment of the disclosure.

FIG. 2 shows a physiological sensor which is a resistor-type sensor.

FIG. 3 shows a physiological sensor which is a photoelectric sensor.

In the various figures of the drawings, the same numerals designate the same or similar parts. Furthermore, when the terms “first”, “second”, “third”, “fourth”, “inner”, “outer”, “top”, “bottom”, “front”, “rear” and similar terms are used hereinafter, it should be understood that these terms have reference only to the structure shown in the drawings as it would appear to a person viewing the drawings, and are utilized only to facilitate describing the disclosure.

### DETAILED DESCRIPTION OF THE INVENTION

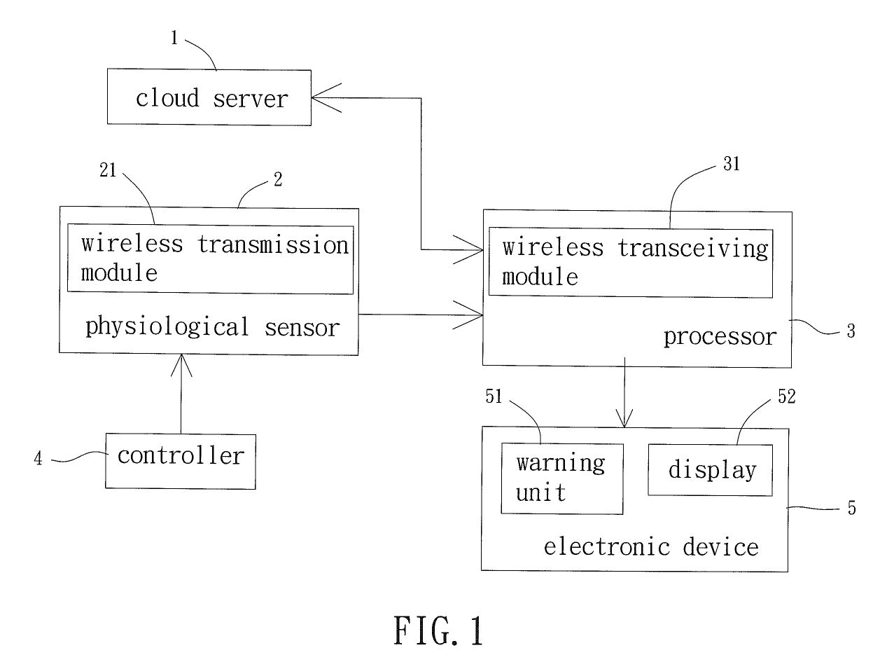

FIG. 1 shows a block diagram of a health care system according to an embodiment of the disclosure. The health care system includes a cloud database 1, a physiological sensor 2 and a processor 3. The processor 3 is electrically connected to the cloud database 1 and the physiological sensor 2.

The cloud database 1 is used to store a reference waveform. The cloud database 1 may be a remote database that can be accessed through a wired or wireless network, such as Dropbox. The generation of the reference waveform is not limited. For example, the physiological sensor 2 can measure a detected part of the body of a healthy person to generate a reference physiological signal. The processor 3 converts the reference physiological signal into the reference waveform, and transmits the reference waveform to the cloud database 1 for storage. The reference waveform in the cloud database 1 may be used for subsequent data comparison. However, the cloud database 1 may store a plurality of reference waveforms. Each reference waveform represents the reference physiological signal of an individual person.

The physiological sensor 2 is used to detect the detected part of the body and to generate a detected physiological signal. The physiological sensor 2 may be a resistor-type sensor or a photoelectric sensor. The physiological sensor 2 may be a three-lead electrocardiogram signal sensor capable of detecting the physiological change between the normal and abnormal ECG signals. The physiological sensor 2 may also be combined with a wearable device so that it can be attached to the user. However, this is not used to limit the disclosure. In addition, the physiological sensor 2 may include a wireless transmission module 21, which can be a WIFI structure, a zigbee structure or a Bluetooth structure. As such, the physiological sensor 2 can be electrically connected to the processor 3 through the wireless transmission module 21, improving the convenience in data transmission.

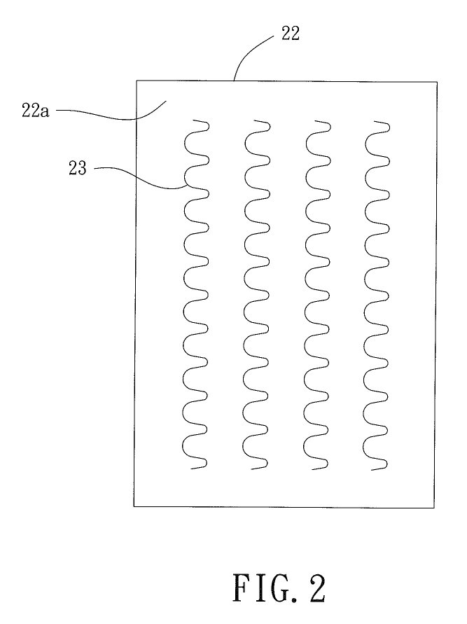

When the physiological sensor 2 is a resistor-type sensor as shown in FIG. 2, the physiological sensor 2 includes a resistor-type substrate 22 and a metal nanowire array 23 in addition to the wireless transmission module 21. The resistor-type substrate 22 includes a sensing face 22a. The metal nanowire array 23 is disposed on the sensing face 22a. The metal nanowire array 23 is used to detect the resistance of the detected part of the body, and the detected physiological signal can be generated based on the resistance. As such, the physiological sensor 2 can generate the detected physiological signal through the detection of the resistance, attaining an efficient generation of the detected physiological signal.

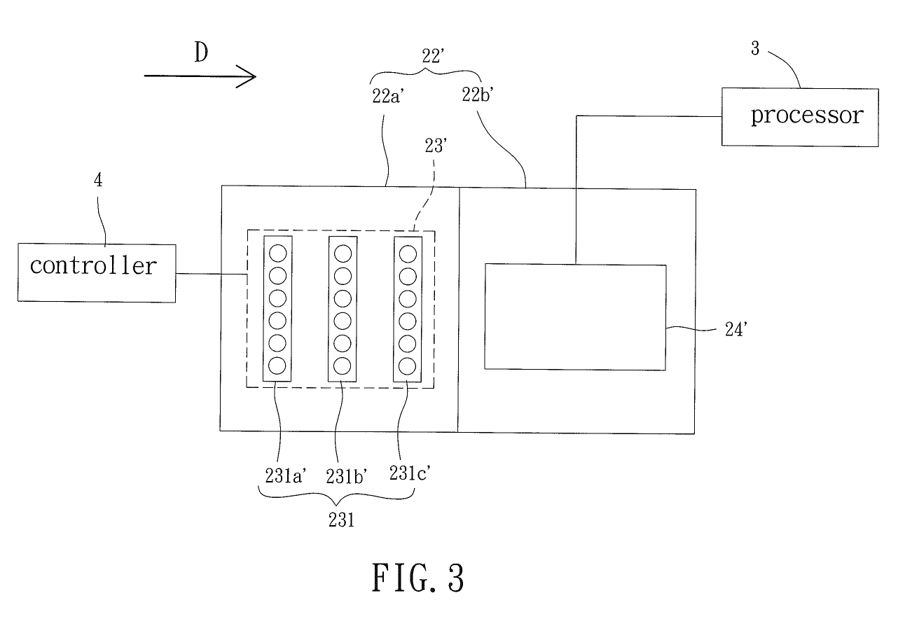

When the physiological sensor 2 is a photoelectric sensor as shown in FIG. 3, the physiological sensor 2 includes a photoelectric substrate 22′, a light emitting unit 23′ and a light detector 24′ in addition to the wireless transmission module 21. The photoelectric substrate 22′ includes a surface that is divided into an emission area 22a′ and a reception area 22b′. The light emitting unit 23′ is arranged on the emission area 22a′ and emits at least one light to the detected part of the body. The light detector 24′ is arranged on the emission area 22a′ and receives at least one reflected light reflected from the detected part of the body. The light detector 24′ generates the detected physiological signal based on the at least one reflected light. As such, the physiological sensor 2 can generate the detected physiological signal through the detection of light, attaining an efficient generation of the detected physiological signal.

When the physiological sensor 2 is a photoelectric sensor, the light emitting unit 23′ may include a plurality of light emitting sections 231′. The plurality of light emitting sections 231′ is arranged on the emission area 22a′ in even intervals along a direction D. The direction D may be parallel to an extending direction of the photoelectric substrate 22′. Each of the plurality of light emitting sections 231′ emits the light with a different wavelength. The quantity of the plurality of light emitting sections 231′ is not limited. In the embodiment, there are three light emitting sections 231′, including a red light emitting section 231a′, a green light emitting section 231b′ and a blue light emitting section 231c′. As such, the light emitting unit 23′ can emit different kinds of lights with different wavelengths. When the light emitting unit 23′ emits lights towards the detected part of the body, although some part of the lights may not be able to penetrate certain tissue in the body due to its wavelength, another part of the lights is still able to reach the detected part. Thus, the detected physiological signal can be efficiently generated.

Each of the plurality of light emitting sections 231′ contains at least one micro light-emitting diode (μLED). The size of the micro LED is 20 μm by 20 μm. Therefore, arrangement of the micro LED not only can reduce the volume and power consumption of the light emitting unit 23′, but also can permit the lights to smoothly reach the detected part due to its finer scale. Thus, the detection accuracy is improved.

Referring to FIG. 1, the processor 3 is electrically connected to the cloud database 1 in order to retrieve the reference waveform therefrom. The processor 3 is also electrically connected to the physiological sensor 2 in order to retrieve the detected physiological signal therefrom. Based on this, the processor 3 converts the detected physiological signal into a physiological waveform, and compares the physiological waveform with the reference waveform to determine a frequency error and a peak error therebetween. When the frequency error is not smaller than a frequency threshold and the peak error is not smaller than a peak threshold, the processor 3 generates a driving signal. The processor 3 may be any processor with a logic calculation function and a statistical analysis function. The processor 3 is able to execute a signal processing procedure, which can convert the detected physiological signal into a waveform signal, as it can be readily appreciated by the person having ordinary skill in the art.

The processor 3 may include a wireless transceiving module 31 electrically connected to the wireless transmission module 21 of the physiological sensor 2. The wireless transceiving module 31 may be a WIFI structure, a zigbee structure or a Bluetooth structure. As such, the processor 3 can be electrically connected to the physiological sensor 2 through the wireless transceiving module 31, improving the convenience in data transmission.

The reference waveform and the physiological waveform may be any physiology-related waveform. In the embodiment, each of the reference waveform and the physiological waveform may be an electrocardiogram (ECG) signal or an electromyogram (EMG) signal. Besides, the reference waveform and the physiological waveform may have the same sensing basis. In the embodiment, the sensing basis includes the testee and the part of the body on which the test is performed. When the reference waveform and the physiological waveform have the same sensing basis, it indicates that the reference waveform and the physiological waveform are measured from the same testee and the same body part. Therefore, the processor 3 can compare the physiological waveform, which is currently retrieved from the testee, with the reference waveform which was retrieved from the same testee when the testee was in a healthy condition. In this regard, the processor 3 can issue the driving signal when there is a large difference between the physiological waveform and the reference waveform. The compared result can be sent to related personnel, increasing the efficiency of health management thereof.

Specifically, the reference waveform was already stored in the cloud database 1, and the physiological sensor 2 can measure the detected part of the body of the testee to generate the reference physiological signal. Therefore, when the processor 3 receives and converts the detected physiological signal into the reference waveform, the processor 3 can retrieve the reference waveform of the detected part of the testee from the cloud database 1. The processor 3 compares the physiological signal with the reference waveform to determine the difference therebetween. Specifically, the processor 3 determines the frequency error and the peak error between the physiological signal and the reference waveform. When the frequency error is not smaller than the frequency threshold and the peak error is not smaller than the peak threshold, the processor 3 generates the driving signal to drive the related electronic device. Thus, the electronic device is able to send a warning message to the user. Since the processor 3 can compare the physiological waveform, which is currently retrieved from the testee, with the reference waveform which was retrieved from the same testee when the testee was in a healthy condition, the processor 3 can issue the driving signal when there is a large difference between the physiological waveform and the reference waveform. The compared result can be sent to related personnel (such as the testee or medical staff), increasing the efficiency of health management thereof.

In the above, the frequency threshold may be about 10% of the frequency value of the reference waveform, and the peak threshold may be about 10% of the peak value of the reference waveform. Under these values, the processor 3 can issue the driving signal when there is a certain difference between the physiological signal and the reference waveform. The compared result can be sent to related personnel (such as the testee or medical staff), increasing the efficiency of health management thereof.

Furthermore, when the frequency error is not smaller than the frequency threshold and the peak error is not smaller than the peak threshold, the processor 3 can also transmit the physiological signal to the cloud database 1 as an abnormal data record. In this arrangement, since the processor 3 can compare the currently-retrieved physiological waveform with the reference waveform previously obtained, the processor 3 can issue the driving signal when there is a large difference between the physiological waveform and the reference waveform. The physiological signal can be sent to the cloud database 1 as the abnormal data record for the reference of the related personnel (such as the testee or medical staff), increasing the efficiency of health management thereof.

Referring to FIGS. 1 and 3, the health care system may further include a controller 4 electrically connected to the light emitting unit 23′. The controller 4 controls the plurality of light emitting sections 231′ to emit different wavelengths of lights in sequence along the direction D. In the embodiment, the plurality of light emitting sections 231′ includes the red light emitting section 231a′, the green light emitting section 231b′ and the blue light emitting section 231c′. Based on this, the controller 4 may control the red light emitting section 231a′, the green light emitting section 231b′ and the blue light emitting section 231c′ to emit lights in sequence. Thus, when the plurality of light emitting sections 231′ emits different wavelengths of lights to the detected part in sequence, the light detector 24′ can sequentially receive the lights reflected from the detected part of the body. As such, the light detector 24′ can generate the detected physiological signal based on the reflected lights. For example, the light detector 24′ can generate the detected physiological signal based a certain wavelength of light, or based on the sequence of the reflected lights. As such, the detection accuracy can be improved.

Alternatively, based on the arrangement of the controller 4, the controller 4 is electrically connected to the light emitting unit 23′, and controls the plurality of light emitting sections 231′ to emit a different wavelength of light in a random manner. In the embodiment, the plurality of light emitting sections 231′ includes the red light emitting section 231a′, the green light emitting section 231b′ and the blue light emitting section 231c′. In this regard, the controller 4 can control the red light emitting section 231a′, the green light emitting section 231b′ and the blue light emitting section 231c′ to emit a red light, a green light or a blue light in a random manner. Based on this, when the plurality of light emitting sections 231′ emits different wavelengths of lights to the detected part of the body in a random manner, the light detector 24′ can receive the lights reflected from the detected part of the body. As such, the light detector 24′ can generate the detected physiological signal based on the reflected lights. For example, the light detector 24′ can generate the detected physiological signal based a certain wavelength of light, or based on the randomness of the reflected lights. As such, the detection accuracy can be improved.

Referring to FIG. 1, the health care system according to the embodiment of the disclosure may further include an electronic device 5. The electronic device 5 can be electrically connected to the processor 3 in order to receive the driving signal therefrom. The electronic device 5 includes a warning unit 51 which issues a warning message upon the reception of the driving signal. The warning unit 51 may be a broadcasting device which broadcasts a warning sound upon the reception of the driving signal. As such, the warning unit 51 of the electronic device 5 can issue the warning message when there is a large difference between the physiological waveform and the reference waveform. The compared result can be sent to related personnel (such as the testee or medical staff), increasing the efficiency of health management thereof.

Based on the arrangement of the electronic device 5, the electronic device 5 is electrically connected to the processor 3 to receive the physiological waveform therefrom. The electronic device 5 includes a display 52 which displays the physiological waveform. As such, the related personnel (such as the testee or medical staff) are able to view the detected result of the physiological sensor 2 via the display 52, improving the efficiency of health management thereof.

Moreover, the electronic device 5 may be a mobile communication device such as a handset or a tablet computer. Based on the arrangement of the warning unit 51 and the display 52, the related personnel (such as the testee or medical staff) are able to know the compared result via the warning unit 51, or to view the compared result via the display 52. Thus, the efficiency of the health management can be improved.

In summary, the health care system according to the embodiment of the disclosure may use the processor 3 to compare the currently-retrieved physiological waveform with the reference waveform previously obtained. In this regard, the processor 3 can issue the driving signal when there is a large difference between the physiological waveform and the reference waveform. The compared result can be sent to related personnel (such as the testee or medical staff), increasing the efficiency of health management thereof.

Although the disclosure has been described in detail with reference to its presently preferable embodiments, it will be understood by one of ordinary skill in the art that various modifications can be made without departing from the spirit and the scope of the disclosure, as set forth in the appended claims.

What is claimed is:

1. A health care system comprising:
a cloud database configured to store a reference waveform and an abnormal data record;
a physiological sensor configured to detect a detected part of a body and to generate a detected physiological signal; and
a processor electrically connected to the cloud database and retrieving the reference waveform from the cloud database, wherein the processor is electrically connected to the physiological sensor to receive the detected physiological signal, wherein the processor converts the detected physiological signal into a physiological waveform, compares the physiological waveform with the reference waveform to determine a frequency error and a peak error therebetween, generates a driving signal when the frequency error is not smaller than a frequency threshold and the peak error is not smaller than a peak threshold, and transmits the physiological signal to the cloud database as the abnormal data record.

2. The health care system as claimed in claim 1, wherein the frequency threshold is substantially 10% of a frequency value of the reference waveform.

3. The health care system as claimed in claim 1, wherein the peak threshold is substantially 10% of a peak value of the reference waveform.

4. The health care system as claimed in claim 1, wherein the physiological sensor is a resistor-type sensor.

5. The health care system as claimed in claim 4, wherein the resistor-type sensor comprises a resistor-type substrate and a metal nanowire array, wherein the resistor-type substrate comprises a sensing face, wherein the metal nanowire array is disposed on the sensing face, wherein the metal nanowire array is used to detect a resistance of the detected part of the body, and the detected physiological signal is generated based on the resistance.

6. The health care system as claimed in claim 1, wherein the physiological sensor is a photoelectric sensor.

7. The health care system as claimed in claim 6, wherein the photoelectric sensor comprises a photoelectric substrate, a light emitting unit and a light detector, wherein the photoelectric substrate comprises a surface that is divided into an emission area and a reception area, wherein the light emitting unit is arranged on the emission area and emits at least one light to the detected part of the body, wherein the light detector is arranged on the emission area and is used to receive at least one reflected light reflected from the detected part of the body, and wherein the light detector generates the detected physiological signal based on the at least one reflected light.

8. The health care system as claimed in claim 7, wherein the light emitting unit comprises a plurality of light emitting sections capable of emitting a plurality of lights with different wavelengths, wherein the plurality of light emitting sections is arranged on the emission area in intervals along a direction.

9. The health care system as claimed in claim 8, further comprising a controller electrically connected to the light emitting unit, wherein the controller is used to control the plurality of light emitting sections to sequentially emit the plurality of lights along the direction.

10. The health care system as claimed in claim 8, further comprising a controller electrically connected to the light emitting unit, wherein the controller is used to control the plurality of light emitting sections to emit the plurality of lights in a random manner.

11. The health care system as claimed in claim 8, wherein a quantity of the plurality of light emitting sections is 3, and the plurality of light emitting sections comprises a red light emitting section, a green light emitting section and a blue light emitting section.

12. The health care system as claimed in claim 8, wherein each of the plurality of light emitting sections comprises at least one micro light-emitting diode, and each of the at least one micro light-emitting diode has a size of 20 μm by 20 μm.

13. The health care system as claimed in claim 1, wherein the physiological sensor further comprises a wireless transmission module, wherein the processor comprises a wireless transceiving module electrically connected to the wireless transmission module.

14. The health care system as claimed in claim 13, wherein each of the wireless transmission module and the wireless transceiving module is a WIFI structure, a zigbee structure or a Bluetooth structure.

15. The health care system as claimed in claim 1, wherein the reference waveform and the physiological waveform have a same sensing basis.

16. The health care system as claimed in claim 1, wherein each of the reference waveform and the physiological waveform is an electrocardiogram signal or an electromyogram signal.

17. The health care system as claimed in claim 1, further comprising an electronic device electrically connected to the processor in order to receive the driving signal, wherein the electronic device comprises a warning unit which issues a warning message upon the reception of the driving signal.

18. The health care system as claimed in claim 1, further comprising an electronic device electrically connected to the processor in order to receive the physiological waveform, wherein the electronic device comprises a display which is used to display the physiological waveform.

19. The health care system as claimed in claim 17, wherein the electronic device is a mobile communication device.

20. The health care system as claimed in claim 1, wherein the physiological sensor is a three-lead ECG signal sensor.
